(19) United States
(12) Patent Application Publication
(10) Pub. No.: US 2009/0222979 A1
(43) Pub. Date: Sep. 10, 2009

(54) Protective Goggle Assembly

(76) Inventor: Ching-Hsiang Wang, Tainan (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/042,409

(22) Filed: Mar. 5, 2008

(51) Int. Cl. A61F 9/02

(52) U.S. Cl. 2/431; 2/452

(57) ABSTRACT

A protective goggle assembly comprises an integrally formed lens, a front rigid frame, a rear resilient frame and a strap having a buckle attached to each of two ends; the rigid frame comprises a middle section having an upper end and a lower end provided with a vertical flange formed with a first hole and a horizontal rim formed with a second hole communicating with the first hole, in which the first and second holes are penetrated by and engaged with bent protrusions formed on the lens; and the rigid frame comprises with a thinner frame portion having notches formed on an outward side, so that grooves formed on the resilient frame can receive the thinner frame portions, while engaging tabs raised inside the grooves of the resilient frame can be engaged in the notches on the thinner frame portions. During assembly, the lens, the rigid frame and the resilient frame can be rapidly engaged, and stably positioned, with one another.

### BACKGROUND OF THE INVENTION

1. Technical Field

The present invention relates to a protective goggle assembly, and more particularly, to a protective goggle assembly comprising an integrally formed lens, a front rigid frame, a rear resilient frame and a strap, wherein the front rigid frame combined with the lens can be rapidly engaged with the rear resilient frame during assembly so as to be stably positioned therewith; the frames installed with the lens can be further buckled with the strap in a simple inserting operation; and the protective goggle assembly can be conveniently disassembled when it is necessary to remove the various components separately.

2. Description of Related Art

Protective goggle assemblies currently designed in the goggle industry usually comprise components each having a predetermined assembly direction designed for better use, so that the components can be rapidly and conveniently assembled and easily disassembled when needed. An example of such protective goggle assemblies comprises a particularly shaped lens having a plurality of engaging portions extending from a periphery of the lens at intervals. These engaging portions are engageable with through holes formed on a matching, resilient frame. The particularly shaped lens further has a nose pad portion at a lower end thereof wherein a rearward projection basing on the nose pad portion can be inserted into a groove formed correspondingly on the frame. In addition, the frame is provided on an upper edge and a lower edge thereof with a plurality of vent holes at intervals. As such, the particularly shaped lens can be rapidly assembled, and therefore maintain stably positioned, with the resilient frame by being engaged therewith. Another example of the aforementioned protective goggle assemblies comprises a particularly shaped lens which is combined with a frame and has an engaging portion on each of two outer sides thereof. The engaging portions allow fastening sections attached to two ends of a strap to penetrate through holes formed on two sides of the frame, so that boundary holes formed at the fastening sections attached to the two ends of the strap are stably positioned with the engaging portions provided on the two outer sides of the particularly shaped lens. As a result, the components can be easily disassembled when the protective goggle assembly is not in use.

### SUMMARY OF THE INVENTION

The present invention provides an alternative design for the assembled configuration of components of prior art protective goggle assemblies, in hope that a front rigid frame combined with an integrally formed lens can be rapidly engaged with a rear resilient frame during assembly so as to be stably positioned therewith; the frames installed with the lens can be rapidly buckled with a strap in a simple inserting operation; and the protective goggle assembly thus formed can be conveniently disassembled when it is necessary to remove the various components separately.

A primary objective of the present invention is to provide a protective goggle assembly comprising an integrally formed lens, a front rigid frame, a rear resilient frame and a strap having a buckle attached to each of two ends thereof. The front rigid frame comprises a middle section having an upper end and a lower end, wherein each of the upper and lower ends is provided with a vertical flange formed with a first hole and a horizontal rim which is adjacent to the vertical flange and formed with a second hole in communication with the first hole. The interconnected first and second holes are penetrated by and engaged with bent protrusions formed respectively on an upper end and a lower end of the lens at locations corresponding to the interconnected first and second holes. The front rigid frame further comprises a rearward section having an upper edge and a lower edge, wherein each of the upper and lower edges is provided with a thinner frame portion having a plurality of notches formed at intervals on an outward side thereof, so that when the rear resilient frame is disposed adjacent enough to a rear side of the front rigid frame, grooves formed respectively on an upper edge and a lower edge of the rear resilient frame at locations corresponding to the thinner frame portions can receive the thinner frame portions, respectively, while each of a plurality of engaging tabs raised inside the grooves formed respectively on the upper and lower edges of the rear resilient frame can be engaged in the corresponding notch on the thinner frame portions. During assembly, the lens, the front rigid frame and the rear resilient frame can be rapidly engaged, and therefore stably positioned, with one another.

A second objective of the present invention is to provide the foregoing protective goggle assembly, wherein each of the upper and lower edges of the rear resilient frame to be assembled with the front rigid frame is provided with a concave portion adjacent to the groove located on the corresponding upper or lower edge of the rear resilient frame, in which the concave portion is in communication with vent holes provided adjacent to the plurality of engaging tabs in said groove.

A third objective of the present invention is to provide the foregoing protective goggle assembly, wherein the front rigid frame is formed with a recess on an outer edge of each of two sides thereof for snuggly receiving an extended section formed on each of two sides of the integrally formed lens at locations corresponding to the recesses.

A fourth objective of the present invention is to provide the foregoing protective goggle assembly, wherein the rear resilient frame to be assembled with the front rigid frame is provided with a through hole at a middle section of each of the two sides thereof, and the integrally formed lens to be assembled to a front side of the front rigid frame is provided with a raised portion on an outer side of each of the extended sections formed on the two sides thereof so that an engaging member formed on an inner side of each of the buckles attached to the two ends of the strap can be passed through the corresponding through hole on the rear resilient frame while an aperture formed on an outer side of each of the buckles can be penetrated by the corresponding raised portion on the lens.

A fifth objective of the present invention is to provide the foregoing protective goggle assembly, wherein the integrally formed lens, the rigid frame and the resilient frame are each made in a different color during a manufacturing process so as to increase a variety of a protective goggle sub-assembly consisting of the lens and the frames.

### BRIEF DESCRIPTION OF THE DRAWINGS

The invention as well as a preferred mode of use, further objectives and advantages thereof will best be understood by reference to the following detailed description of an illustrative embodiment when read in conjunction with the accompanying drawings, wherein:

FIG. 1 is a perspective exploded view of a lens and a rigid frame according to the present invention;

FIG. 2 is a schematic assembled view of the lens and the rigid frame according to the present invention;

FIG. 3 is a cross-sectional view of the sub-assembly of the lens and the rigid frame according to the present invention;

FIG. 4 is an exploded view of the rigid frame and a resilient frame according to the present invention;

FIG. 5 is a schematic assembled view of the rigid frame and the resilient frame according to the present invention;

FIG. 6 is a cross-sectional view of the sub-assembly of the rigid frame and the resilient frame according to the present invention;

FIG. 7 is a perspective exploded view of a protective goggle sub-assembly and a strap according to the present invention; and

FIG. 8 illustrates an appearance of the protective goggle sub-assembly buckled with a strap according to the present invention.

### DETAILED DESCRIPTION OF THE PREFERRED EMBODIMENT

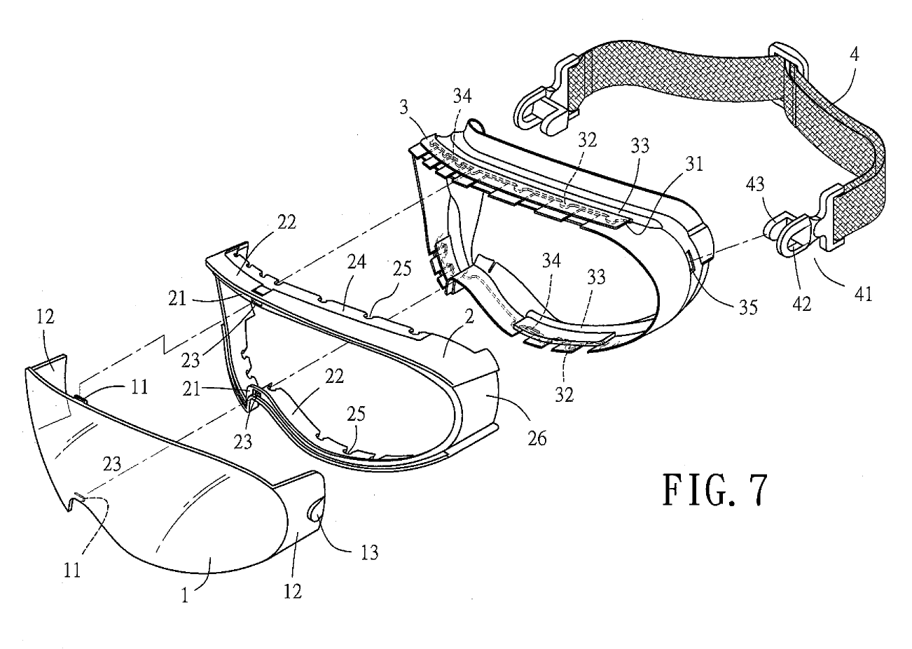

Referring to FIG. 7, a protective goggle assembly according to the present invention comprises an integrally formed lens 1, a rigid frame 2, a resilient frame 3 and a strap 4 having a buckle 41 attached to each of two ends thereof.

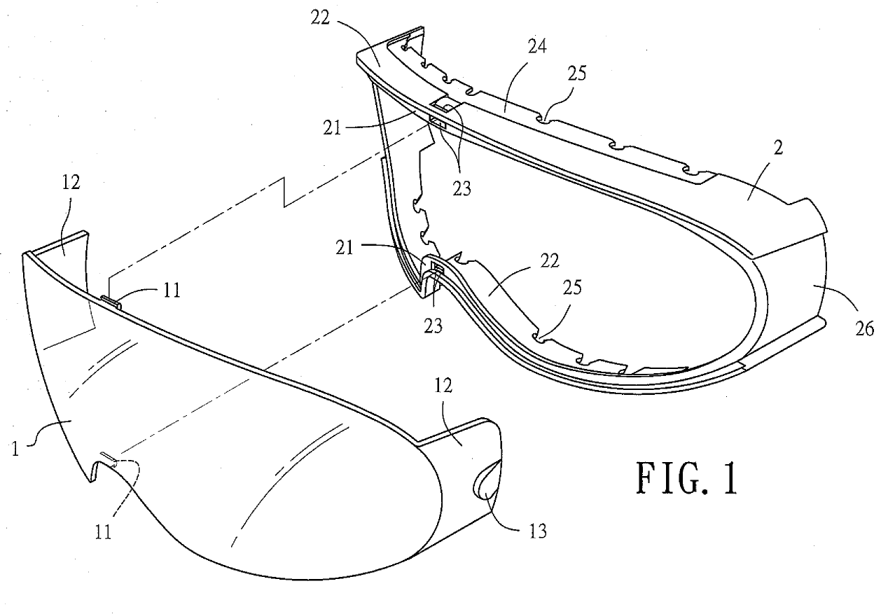

The integrally formed lens 1 comprises a middle section having an upper end and a lower end, wherein each of the upper and lower ends is provided with a bent protrusion 11 (also shown in FIG. 1). The downwardly and upwardly bent protrusions 11 are correspondingly formed towards respective assembly directions thereof. In addition, the lens 1 is further provided with a rearward extended section 12 on each of two sides thereof, wherein a raised portion 13 is formed on an outer side of each of the extended sections 12.

The rigid frame 2 comprises a middle section having an upper end and a lower end, wherein each of the upper and lower ends is provided with a vertical flange 21 and a horizontal rim 22 adjacent to the vertical flange 21, in which the adjacent vertical flange 21 and horizontal rim 22 are each formed with a hole 23 that is in communication with each other (referring also to FIG. 1). Furthermore, the rigid frame 2 comprises a rearward section having an upper edge and a lower edge, wherein each of the upper and lower edges is provided with a thinner frame portion 24 having a plurality of notches 25 formed at intervals on an outward side thereof. Moreover, the rigid frame 2 has a recess 26 on an outer edge of each of two sides thereof.

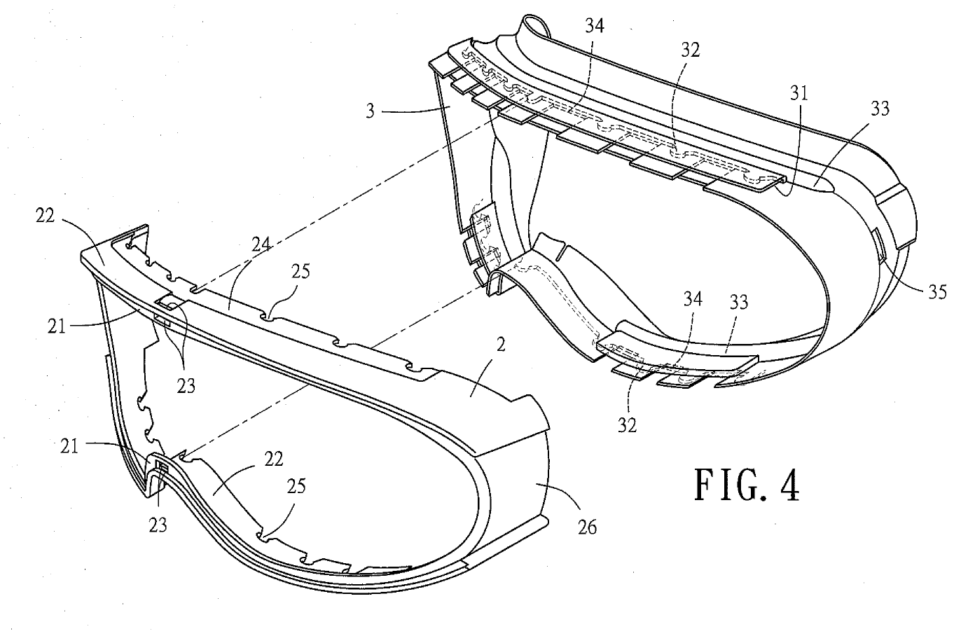

The resilient frame 3 comprises a forward upper edge and a forward lower edge which are each formed with a groove 31 corresponding to the thinner frame portions 24 of the rigid frame 2 (also referring to FIG. 4). Each of the grooves 31 formed on the upper and lower edges of the resilient frame 3 has a plurality of engaging tabs 32 raised inside said groove 31 at intervals. In addition, a concave portion 33 is formed at a middle section of each of the upper and lower edges of the resilient frame 3, adjacent to the corresponding groove 31 formed on the upper or lower edge of the resilient frame 3, wherein the concave portion 33 is in communication with vent holes 34 provided adjacent to the plurality of engaging tabs 32 extending from within said groove 31. In addition, the resilient frame 3 has a through hole 35 at a middle section of each of two sides thereof.

The strap 4 has the buckle 41 attached to each of the two ends thereof, wherein each of the buckles 41 comprises an engaging member 42 formed on an inner side thereof and an aperture 43 formed on an outer side thereof.

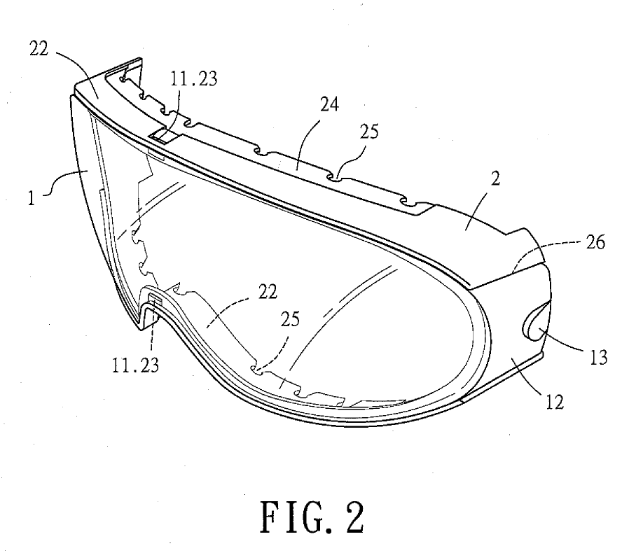

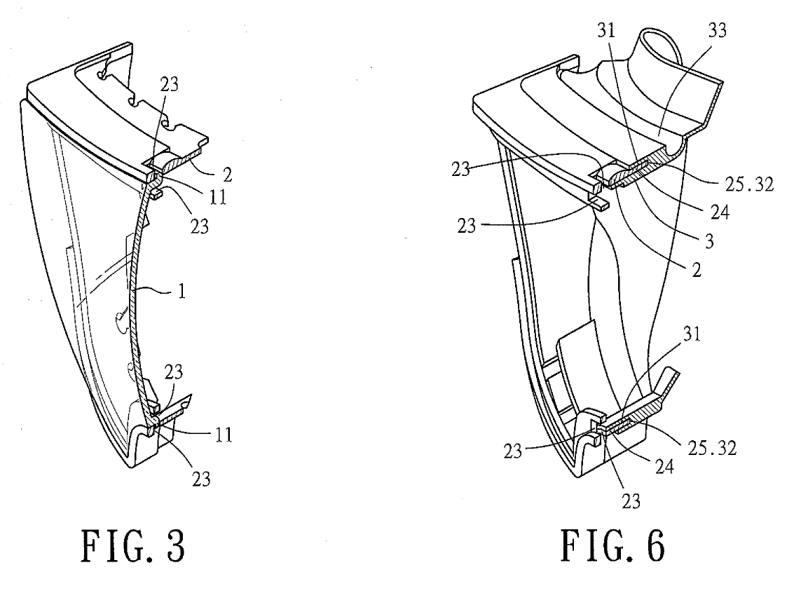

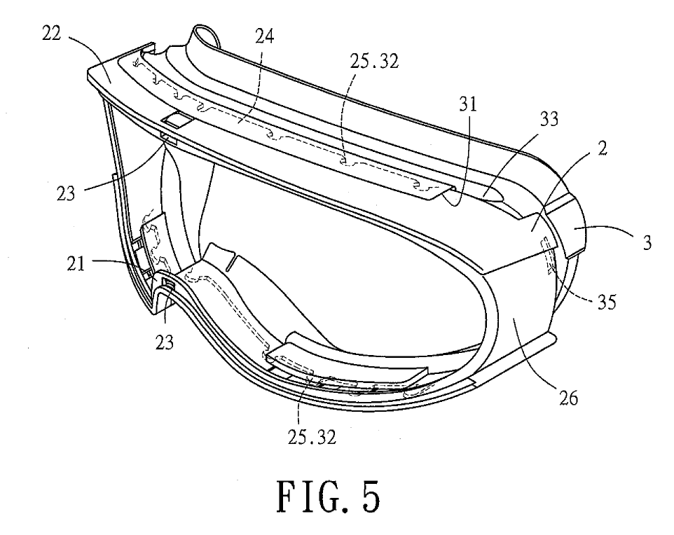

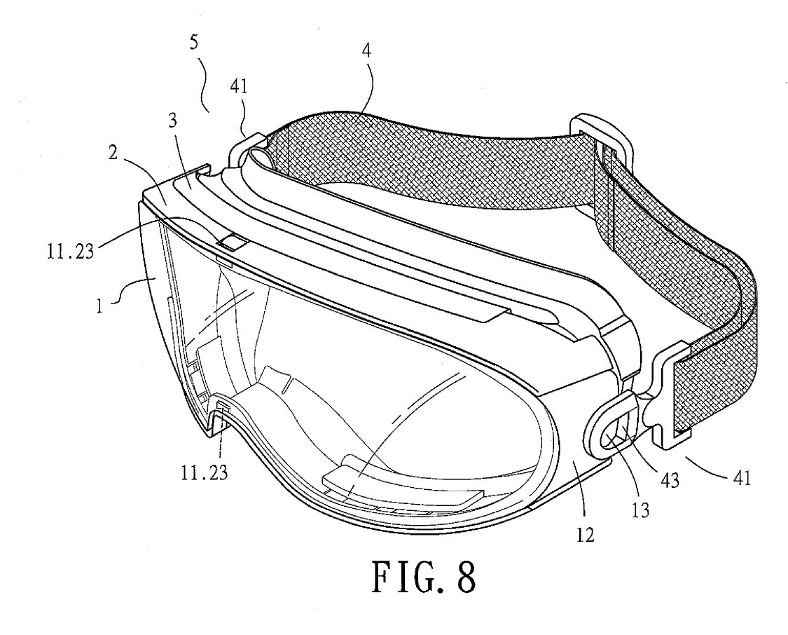

Referring to FIGS. 1 and 3, in order to assemble the integrally formed lens 1 with the rigid frame 2, the bent protrusions 11 extending from the upper and lower ends of the middle section of the lens 1 are respectively passed through and engaged with the holes 23 formed at the corresponding locations on the upper and lower ends of the rigid frame 2 (as shown in FIG. 3), and the rearward extended sections 12 basing on the two sides of the integrally formed lens 1 are snuggly received, respectively, in the recesses 26 formed at the corresponding locations on the two sides of the rigid frame 2, as shown in FIG. 2. Now referring to FIGS. 4 and 6, in order to assemble the rigid frame 2 with the resilient frame 3, the thinner frame portions 24 formed at the upper and lower edges of the rearward section of the rigid frame 2 are inserted into the corresponding grooves 31 formed at the upper and lower edges of the resilient frame 3 (as shown in FIG. 6), so that the plurality of engaging tabs 32 raised inside the upper and lower grooves 31 of the resilient frame 3 are engaged respectively in the notches 25 of the thinner frame portions 24, as shown in FIG. 5. Thus, the integrally formed lens 1, the front rigid frame 2 and the rear resilient frame 3 can be rapidly engaged, and therefore stably positioned, with one another, forming a protective goggle sub-assembly 5 shown in FIG. 8.

Referring to FIGS. 7 and 8, when the protective goggle sub-assembly 5 composed of the integrally formed lens 1, the rigid frame 2 and the resilient frame 3 is to be assembled with the strap 4, the engaging members 42 formed on the inner sides of the buckles 41 attached to the two ends of the strap 4 are inserted into the through holes 35 on the resilient frame 3 while the apertures 43 formed on the outer sides of the buckles 41 are penetrated by the raised portions 13 formed on the extended sections 12 on the two sides of the lens 1 (as shown in FIG. 8), so that the strap 4 is easily engaged, and therefore accurately positioned, with the protective goggle sub-assembly 5 and can be conveniently removed from the sub-assembly 5 when not in use.

The integrally formed lens 1, the rigid frame 2 and the resilient frame 3 can be each made in a different color during a manufacturing process so as to increase a variety of the protective goggle sub-assembly 5.

What is claimed is:

1. A protective goggle assembly, which comprises an integrally formed lens, a front rigid frame, a rear resilient frame and a strap having a buckle combined to each of two ends thereof, and is characterized in that:
the front rigid frame comprises a middle section having an upper end and a lower end, wherein each of the upper and lower ends is provided with a vertical flange formed with a first hole and a horizontal rim which is adjacent to the vertical flange and formed with a second hole in communication with the first hole, in which the interconnected first and second holes are penetrated by and engaged with bent protrusions formed respectively on an upper end and a lower end of the lens at locations corresponding to the interconnected first and second holes; and
the front rigid frame further comprises a rearward section having an upper edge and a lower edge, wherein each of the upper and lower edges is provided with a thinner frame portion having a plurality of notches formed at intervals on an outward side thereof, so that when the rear resilient frame is placed adjacent enough to a rear side of the rigid frame, grooves formed respectively on an upper edge and a lower edge of the rear resilient frame at locations corresponding to the thinner frame portions can receive the thinner frame portions, respectively, while each of a plurality of engaging tabs raised inside the grooves formed respectively on the upper and lower edges of the rear resilient frame can be engaged in the corresponding notch on the thinner frame portions;
whereby during assembly, the integrally formed lens, the front rigid frame and the rear resilient frame can be rapidly engaged, and therefore stably positioned, with one another.

2. The protective goggle assembly as claimed in claim 1, wherein each of the upper and lower edges of the rear resilient frame to be assembled with the front rigid frame is provided with a concave portion adjacent to the groove located on the corresponding upper or lower edge of the rear resilient frame, in which the concave portion is in communication with vent holes provided adjacent to the plurality of engaging tabs in said groove.

3. The protective goggle assembly as claimed in claim 1, wherein the front rigid frame is formed with a recess on an outer edge of each of two sides thereof for snuggly receiving an extended section formed on each of two sides of the integrally formed lens at locations corresponding to the recesses.

4. The protective goggle assembly as claimed in claim 1, wherein the rear resilient frame to be assembled with the front rigid frame is provided with a through hole at a middle section of each of the two sides thereof, and the integrally formed lens to be assembled to a front side of the front rigid frame is provided with a raised portion on an outer side of each of the extended sections formed on the two sides thereof, so that an engaging member formed on an inner side of each of the buckles attached to the two ends of the strap can be passed through the corresponding through hole on the rear resilient frame while an aperture formed on an outer side of each of the buckles can be penetrated by the corresponding raised portion on the lens.

5. The protective goggle assembly as claimed in claim 1, wherein the integrally formed lens, the rigid frame and the resilient frame are each made in a different color during a manufacturing process.
